(19) United States
(12) Patent Application Publication
(10) Pub. No.: US 2011/0277537 A1
(43) Pub. Date: Nov. 17, 2011

(54) ALCOHOL DRINKING STATE CHECK DEVICE

(75) Inventors: Hanzo Tsuzuki, Shizuoka (JP); Tetsuya Sugimoto, Shizuoka (JP); Hiroaki Takagi, Tokyo (JP)

(73) Assignee: TOKAI DENSHI, INC., Fuji-shi (JP)

(21) Appl. No.: 13/103,313

(22) Filed: May 9, 2011

(30) Foreign Application Priority Data
May 11, 2010 (JP) 2010-109469

(51) Int. Cl. G01N 33/497

(52) U.S. Cl. 73/23.3

(57) ABSTRACT

A to-be-called side device includes an alcohol drinking state check means for performing an alcohol drinking state check of a user, a peripheral state obtaining means for obtaining peripheral states by sound and image to keep an illegal communication as to the check performed between a person who monitors the check and the user as evidence, a temporary record continuation means for continuing the temporary record of the peripheral states obtained by the peripheral state obtaining means, and a pre-check/post-check record means for recording the peripheral state for 10 minutes before the check and the peripheral state for 10 minutes after the check in the respective checks, wherein the pre-check/post-check record means obtains the record of the peripheral state at least before the check from the record by the temporarily record continuation means in the record of the peripheral states for 20 minutes before and after the check.

### BACKGROUND OF THE INVENTION

1. Field of the Invention

The invention relates to an alcohol drinking state check device capable of performing an alcohol drinking state check of a user.

2. Description of the Related Art

As a conventional alcohol drinking state check device, there is known an alcohol detection system provided with a digital camera for picking up the image of a driver whose alcohol concentration in a breath is being measured to prevent the measurement of alcohol concentration in a breath of an imposter who drinks no alcohol (refer to, for example, Patent Document 1)
Patent document 1: Jpn. Pat. Appln. KOKAI Publication No. 2005-157599

An alcohol concentration in a breath of a driver who drives a vehicle in business is measured before the driver drives the vehicle to prevent drink-driving. However, various illegal measurements are performed by a driver who drinks alcohol such as an illegal measurement by an imposter who drinks no alcohol. In a worst case, an illegal measurement may be performed in conspiracy of a driver with a person in charge of monitoring the measurement by a driver. In the conspiracy, a communication is made between the driver and the person in charge as to the illegal measurement in many cases.

However, the conventional alcohol drinking state check device cannot keep the communication described above as evidence although the device can pick up the image of a driver whose alcohol concentration in a breath is being measured.

To cope with the above problem, an object of the invention is to provide an alcohol drinking state check device capable of keeping a communication as to illegality for an alcohol drinking state check as evidence.

### SUMMARY OF THE INVENTION

An alcohol drinking state check device of the invention is characterized by including an alcohol drinking state check means for performing an alcohol drinking state check of a user, a peripheral state obtaining means for obtaining a peripheral state by at least one of sound and image to keep a communication as to the check which is illegal between a person who monitors the check performed by the user and the user as evidence, a temporary record continuation means for continuing the temporary record of the peripheral states obtained by the peripheral state obtaining means, and a pre-check/post-check record means for recording the peripheral states for a predetermined time before and after the check in the respective checks, wherein the pre-check/post-check record means obtains the record of the peripheral state at least before the check from the record by the temporarily record continuation means in the record of the peripheral states for the predetermined time before and after the check.

With the configuration, since the alcohol drinking state check device of the invention records the peripheral states for the predetermined time before and after the alcohol drinking state check is performed by the user in the respective checks, when a communication as to illegality for the alcohol drinking state check is performed in the predetermined time before and after the alcohol drinking state check, the communication can be kept as the evidence.

Further, the alcohol drinking state check device of the invention preferably includes an external transmission means for transmitting the peripheral states recorded by the pre-check/post-check record means to the outside.

With the configuration, since the alcohol drinking state check device of the invention transmits the evidence to the outside, the evidence can be stored in the external device. Accordingly, the alcohol drinking state check device of the invention can make it difficult to illegally erase the evidence in comparison with the case that the evidence is stored only in the alcohol drinking state check device itself.

An alcohol drinking state check program for causing a computer to execute a process stored by a non-transitory computer-readable storage medium of the invention is characterized by including an alcohol drinking state check step of performing an alcohol drinking state check of a user, a peripheral state obtaining step of obtaining a peripheral state by at least one of sound and image to keep a communication as to the check which is illegal between a person who monitors the check performed by the user and the user as evidence, a temporary record continuation step of continuing the temporary record of the peripheral states obtained by the peripheral state obtaining step, and a pre-check/post-check record step of recording the peripheral states for a predetermined time before and after the check in the respective checks, wherein the pre-check/post-check record step obtains the record of the peripheral state at least before the check from the record by the temporarily record continuation step in the record of the peripheral states for the predetermined time before and after the check.

With the configuration, since the computer which executes the alcohol drinking state check program stored by the non-transitory computer-readable storage medium of the invention records the peripheral states for the predetermined time before and after the alcohol drinking state check of the user in the respective checks, the communication as to illegality for the alcohol drinking state check can be kept as the evidence.

Further, the alcohol drinking state check program of the invention preferably includes an external transmission step of transmitting the peripheral states recorded by the pre-check/post-check record step to the outside.

An alcohol drinking state check method of the invention is characterized by including an alcohol drinking state check step of performing an alcohol drinking state check of a user, a peripheral state obtaining step of obtaining a peripheral state by at least one of sound and image to keep a communication as to the check which is illegal between a person who monitors the check performed by the user and the user as evidence, a temporary record continuation step of continuing the temporary record of the peripheral states obtained by the peripheral state obtaining step, and a pre-check/post-check record step of recording the peripheral states for a predetermined time before and after the check in the respective checks, wherein the pre-check/post-check record step obtains the record of the peripheral state at least before the check from the record by the temporarily record continuation step in the record of the peripheral states for the predetermined time before and after the check.

Further, the alcohol drinking state check method of the invention preferably includes an external transmission step of transmitting the peripheral states recorded by the pre-check/post-check record step to the outside.

The alcohol drinking state check device of the invention can keep the communication as to illegality of the alcohol drinking state check as the evidence.

### BRIEF DESCRIPTION OF THE DRAWINGS

FIG. 1 is a block diagram of a roll call system according to an embodiment of the invention;

FIG. 2 is a block diagram of a call side device shown in FIG. 1;

FIG. 3 is a block diagram of a to-be-called side device shown in FIG. 1;

FIG. 4 is a view showing an example of a call screen displayed on a monitor of the call side device shown in FIG. 1;

FIG. 5 is a view showing an example of a list screen displayed on a monitor of the to-be-called side device shown in FIG. 1;

FIG. 6 is a flowchart of an operation of the to-be-called side device shown in FIG. 1 when a call is requested;

FIG. 7 is a view showing an example of queries displayed on the monitor of the to-be-called side device shown in FIG. 1;

FIG. 8 is a flowchart of an operation of the call side device shown in FIG. 1, the call side device being a server when a to-be-called person is registered to a call waiting list;

FIG. 9 is a flowchart of an operation of the to-be-called side device shown in FIG. 1 when evidence is obtained.

FIG. 10 is a flowchart of an operation of the call side device shown in FIG. 1 when a call is performed; and

FIG. 11 is a block diagram of the to-be-called side device shown in FIG. 1 when a computer is composed of a mobile communication device.

### DESCRIPTION OF THE PREFERRED EMBODIMENT

An embodiment of the invention will be explained below using the drawings.

First, a configuration of a roll call system according to the embodiment of the invention will be explained.

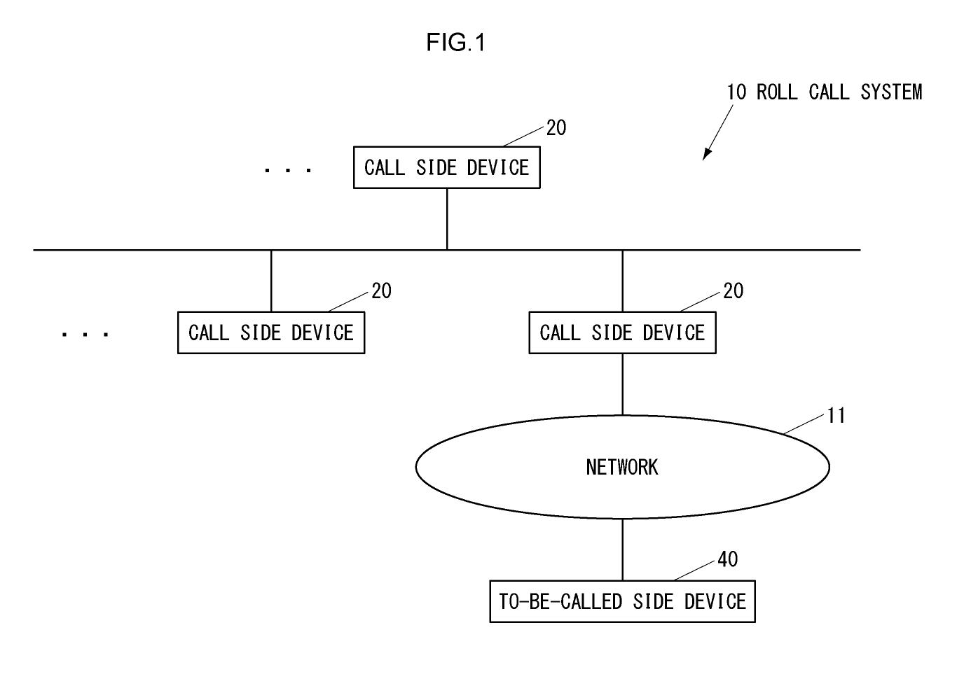

FIG. 1 is a block diagram of a roll call system 10 according to the embodiment.

As shown in FIG. 1, the roll call system 10 includes a call side device 20 used by a call manager who performs a call and a to-be-called side device 40 as an alcohol drinking state check device of the invention which is used by a to-be-called person to whom a call is performed and transmits the behavior of the to-be-called person to the call side device 20 by sound and image. The call side device 20 and the to-be-called side device 40 can communicate with each other via a network 11 such as the Internet and the like. The roll call system 10 includes plural sets of the call side devices 20. Further, although only one set of the to-be-called side device 40 is illustrated in FIG. 1, the roll call system 10 also includes plural sets of the to-be-called side devices 40.

The roll call system 10 is a system used by a carrier and is a system for supporting a call of a truck driver before the driver drives a truck. The call side devices 20 are disposed in a head office of the carrier. The to-be-called side devices 40 are installed to the branch offices of the carrier which are positioned at, for example, several hundreds of locations throughout the country.

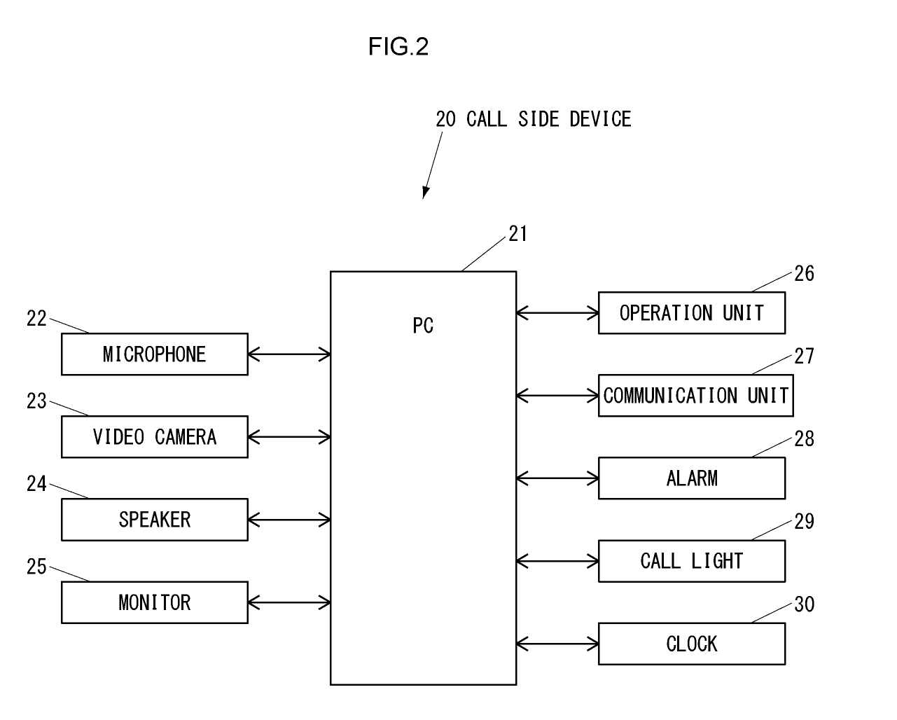

FIG. 2 is a block diagram of the call side device 20.

As shown in FIG. 2, the call side device 20 includes a PC (Personal Computer) 21 as a computer for controlling the call side device 20 in its entirety, a microphone 22 for inputting sound, a video camera 23 for inputting image, a speaker 24 for outputting sound, a monitor 25 for outputting image, an operation unit 26 such as a keyboard, a mouse, a touch panel, to which an operation performed by a call manager is input, a communication unit 27 for making a communication with an external device, an alarm 28 for issuing an alarm by sound, a call light 29 for calling the call manager by light, and a clock 30 for measuring time. Note that the call side device 20 may be configured to substitute the speaker 24 and the monitor 25 for the alarm 28 and the call light 29, respectively.

The PC 21 is composed of a CPU (Central Processing Unit), a ROM (Read Only Memory) and a HDD (Hard Disk Drive) which previously store programs and various types of data, a RAM (Random Access Memory) used as a working area of the CPU, and the like. The CPU is an arithmetic processing unit for operating the PC 21 by executing the programs stored in the ROM and the HDD. When the programs are executed by the CPU, the RAM temporarily stores the programs and various types of data.

In the call side devices 20, one of the call side devices 20 acts as a server, and the remaining call side devices 20 act as clients. Only the PC 21 of the call side device 20 acting as the server stores and manages a call waiting list which is a list of to-be-called persons who wait for a call. The PCs 21 of the call side devices 20 which are the clients do not store the call waiting list and are configured to read and edit the call waiting list stored in the PC 21 of the call side device 20 acting as the server. That is, all the call side devices 20 including the call side device 20 acting as the server use the call waiting list collectively. With the configuration, when calls are performed by plural call side devices 20 at the same time, a disadvantage that the same to-be-called person is called by plural call side devices 20 can be prevented from being occurred.

Likewise, the PC 21 of the call side device 20 acting as the server stores the face images previously registered to the respective to-be-called persons, the past histories previously registered to the respective to-be-called persons, and a standard call time previously set as a time necessary to a call of a to-be-called person. Further, the PC 21 of the call side device 20 acting as the server stores various types of information transmitted from the to-be-called side devices 40 together with a call request. The information is also used by all the call side devices 20 collectively.

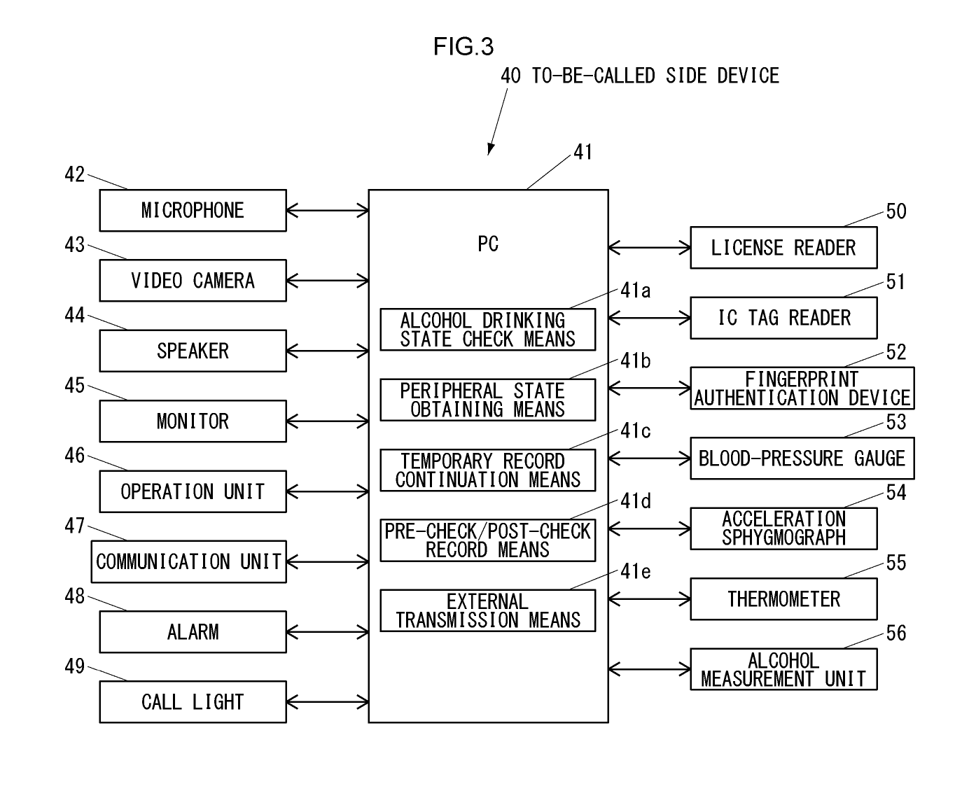

FIG. 3 is a block diagram of a to-be-called side device 40.

As shown in FIG. 3, the to-be-called side device 40 includes a PC 41 as a computer for controlling the to-be-called side device 40 in its entirety, a microphone 42 for inputting sound, a video camera 43 for inputting image, a speaker 44 for outputting sound, a monitor 45 for outputting image, an operation unit 46 such as a keyboard, a mouse, a touch panel, to which an operation performed by a to-be-called person is input, a communication unit 47 for making a communication with an external device, an alarm 48 for issuing an alarm by sound, a call light 49 for calling a to-be-called person by light, a license reader 50 for reading information from the license of a to-be-called person, an IC tag reader 51 for reading information from an IC tag carried by a to-be-called person, a fingerprint authentication device 52 for reading and authenticating the fingerprint of a to-be-called person, a blood-pressure gauge 53 for measuring the blood-pressure of a to-be-called person, an acceleration sphygmograph 54 for measuring the acceleration pulse wave of a to-be-called person, a thermometer 55 for measuring the temperature of a to-be-called person, and an alcohol measurement unit 56 for measuring the alcohol concentration in the breath of a to-be-called person. The IC tag may be buried in, for example, a key holder and attached to a key of a truck by the key holder. Note that the to-be-called side device 40 may be configured to substitute the speaker 44 and the monitor 45 for the alarm 48 and the call light 49, respectively. Further, although the authentication by fingerprint is employed in the embodiment as an authentication method, an authentication by venous, iris, face, and the like other than fingerprint may be employed.

The PC 41 is composed of a CPU, a ROM and a HDD in which programs including an alcohol drinking state check program of the invention and various types of data are previously stored, and a RAM used as a working area of the CPU, and the like. The CPU is an arithmetic processing unit for operating the PC 41 by executing the programs stored in the ROM and the HDD. When the programs are executed by the CPU, the RAM temporarily stores the programs and various types of data. The alcohol drinking state check program may be stored in a portable non-transitory computer-readable storage medium such as a CD-ROM, a DVD disk, and the like and be executed by the PC 41.

Note that a PC 41 is configured to function as an alcohol drinking state check means 41a for performing an alcohol drinking state check (measurement of an alcohol concentration) of a to-be-called person as a user by the alcohol measurement unit 56, a peripheral state obtaining means 41b for obtaining a peripheral state by sound and image using the microphone 42 and the video camera 43, a temporary record continuation means 41c for continuing the temporary record of the peripheral states obtained by the peripheral state obtaining means 41b, a pre-check/post-check record means 41d for recording peripheral states for 10 minutes before and 10 minutes after a check to a non-volatile memory as evidence in the respective checks, and an external transmission means 41e for transmitting the peripheral states recorded by the pre-check/post-check record means 41d to the call side devices 20 as external devices by the communication unit 47. The time of 10 minutes is only an example and the time is not limited to the 10 minutes. Further, a recording time may be different before and after the check.

The peripheral state obtaining means 41b is configured to obtain the peripheral states by the microphone 42 and the video camera 43 at all times while the PC 41 operates.

Further, the temporarily record continuation means 41c is configured to continuously overwrite and store the peripheral states obtained by the peripheral state obtaining means 41b to and in a ring buffer of 30 minutes at all times while the PC 41 operates. Note that the time of the 30 minutes is only an example and the time is not limited to the 30 minutes.

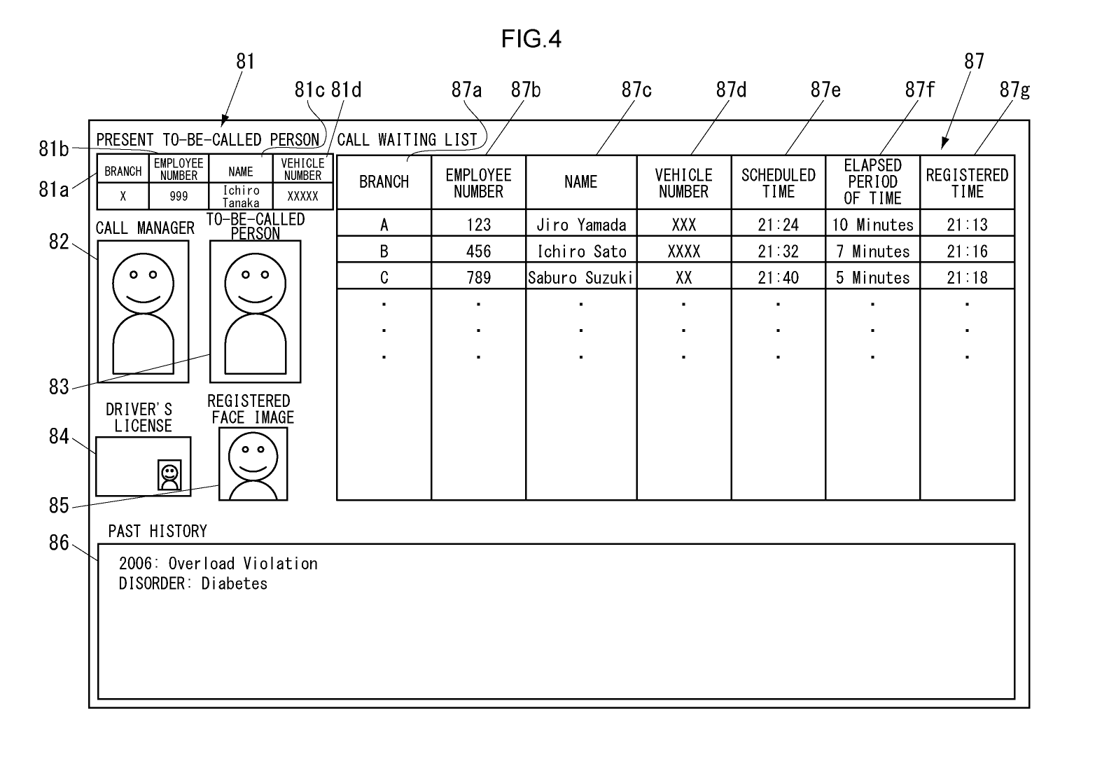

FIG. 4 is a view showing an example of a call screen displayed on the monitor 25 of the call side device 20.

As shown in FIG. 4, the call screen includes a “present to-be-called person” region 81 showing information of a to-be-called person who is being called at present, a “call manager” region 82 showing the image of the call manager, a “to-be-called person” region 83 showing the image of a to-be-called person who is being called at present, a “driver's license” region 84 showing the image of the driver's license of a to-be-called person who is being called at present, a “registered face image” region 85 showing the previously registered face image of a to-be-called person who is being called at present, a “past history” region 86 showing the previously registered past history of a to-be-called person who is being called at present, and a “call waiting list” region 87 showing a call waiting list.

The “present to-be-called person” region 81 includes a “branch” region 81a showing the identification information of a to-be-called side device 40 which requested the call of a to-be-called person who is being called at present, an “employee number” region 81b showing the employee number of a to-be-called person who is being called at present, a “name” region 81c showing the name of a to-be-called person who is being called at present, and a “vehicle number” region 81d showing the vehicle number of a vehicle which is driven by a to-be-called person who is being called at present. The vehicle number is the register number of a vehicle, a number uniquely allocated to a vehicle owned by a firm, and the like. Note that the information shown in the “branch” region 81a, the “employee number” region 81b, the “name” region 81c, and the “vehicle number” region 81d is the identification information of a to-be-called person, and when the PC 21 of the call side device 20 acting as the server receives a call request from a to-be-called side device 40, the identification information is transmitted from the to-be-called side device 40 together with the call request.

The image shown in the “call manager” region 82 is an image picked up by the video camera 23.

The image shown in the “to-be-called person” region 83 is an image picked up by the video camera 43 of the to-be-called side device 40 and transmitted from the to-be-called side device 40 to the call side device 20.

The image shown in the “driver's license” region 84 is an image, which is transmitted, when the PC 21 of the call side device 20 acting as the server receives the call request from the to-be-called side device 40, together with the call request from the to-be-called side device 40. The face image of a to-be-called person in the image shown in the “driver's license” region 84 is used to cause the call manager to determine whether or not the person shown in the “to-be-called person” region 83 is truly the to-be-called person himself or herself.

The face image shown in the “registered face image” region 85 is previously registered in the PC 21 of the call side device 20 acting as the server for the respective to-be-called persons. The face image of a to-be-called person shown in the “registered face image” region 85 is used to cause the call manager to determine whether or not the person shown in the “to-be-called person” region 83 is truly the to-be-called person himself or herself.

The past histories shown in the “past history” region 86 are previously registered in the PC 21 of the call side device 20 acting as the server for the respective to-be-called persons. The past histories include, for example, the past traffic violations of the to-be-called persons, the disorders of the to-be-called persons, and the past work histories of the to-be-called persons.

The respective rows of the call waiting list shown in the “call waiting list” region 87 show information of the respective to-be-called persons who wait for a call, and calls are sequentially performed from an upper row. The respective columns of the call waiting list show a “branch” region 87a showing identification information of the to-be-called side device 40 which requests calls of to-be-called persons, an “employee number” region 87b showing the employee numbers of to-be-called persons, a “name” region 87c showing the names of to-be-called persons, a “vehicle number” region 87d showing the numbers of the vehicles which are driven by to-be-called persons, a “scheduled time” region 87e showing the scheduled times at which calls are performed to to-be-called persons, an “elapsed period of time” region 87f showing the elapsed period of times from the times at which to-be-called persons were registered to the call waiting list to the present time, and a “registered time” region 87g showing the times at which to-be-called persons were registered to the call waiting list. The information shown in the “branch” region 87a, the “employee number” region 87b, the “name” region 87c, and the “vehicle number” region 87d are the identification information of the to-be-called persons, and when the PC 21 of the call side device 20 acting as the server receives a call request from a to-be-called side device 40, the identification information is transmitted from the to-be-called side device 40 together with the call request.

The scheduled time in the “scheduled time” region 87e is information which can be calculated by the PC 21 based on the standard call time previously set as a time necessary to a call of a to-be-called person, the number of the call side devices 20, and the order of the to-be-called persons in the call waiting list. The scheduled time is updated at real time. A standard waiting time is determined as a scheduled waiting time of a to-be-called person in the overall roll call system 10 based on the standard call time and the number of the call side devices 20. For example, in an example shown in FIG. 4, the standard waiting time is 8 minutes, the call of Mr. Ichiro Tanaka as a to-be-called person who is being called at present starts at 21:16, the scheduled call time of Mr. Jiro Yamada who is listed first in the call waiting list is 21:24 which is 8 minutes after the above call time, the scheduled call time of Mr. Ichiro Sato who is listed second in the call waiting list is 21:32 which is further 8 minutes after the above call time, and the scheduled call time of Mr. Saburo Suzuki who is listed third in the call waiting list is 21:40 which is further 8 minutes after the above call time.

Note that the standard waiting time is in inverse proportion to the number of calls which can be processed at the same time. The number of calls which can be processed at the same time is equal to the number of call managers who perform a call, that is, equal to the number of the call side devices 20. Accordingly, the standard waiting time is in inverse proportion to the number of the call side devices 20. That is, the standard waiting time when 2 sets of the call side devices 20 are available is one half that when 1 set of the call side device 20 is available, and the standard waiting time when 3 sets of the call side devices 20 are available is one third that when 1 set of the call side device 20 is available.

When a call is delayed because many to-be-called persons are registered in the call waiting list, a carrier employing the roll call system 10 can overcome the delay by additionally employing the number of the call side devices 20 and the number of the call managers who use the call side devices 20.

The elapsed period of time shown in the “elapsed period of time” region 87f is information which can be calculated based on the registered time shown in the “registered time” region 87g and the present time which can be obtained from the clock 30. The elapsed period of time is updated at real time. For example, in the example shown in FIG. 4, the present time is 21:23, the elapsed period of time of Mr. Jiro Yamada who is listed first in the call waiting list is 10 minutes from the registered time of 21:13, the elapsed period of time of Mr. Ichiro Sato who is listed second in the call waiting list is 7 minutes from the registered time of 21:16, and the elapsed period of time of Mr. Saburo Suzuki who is listed third in the call waiting list is 5 minutes from the registered time of 21:18.

The registered time shown in the “registered time” region 87g is the present time of the clock 30 when a to-be-called person is registered to the call waiting list.

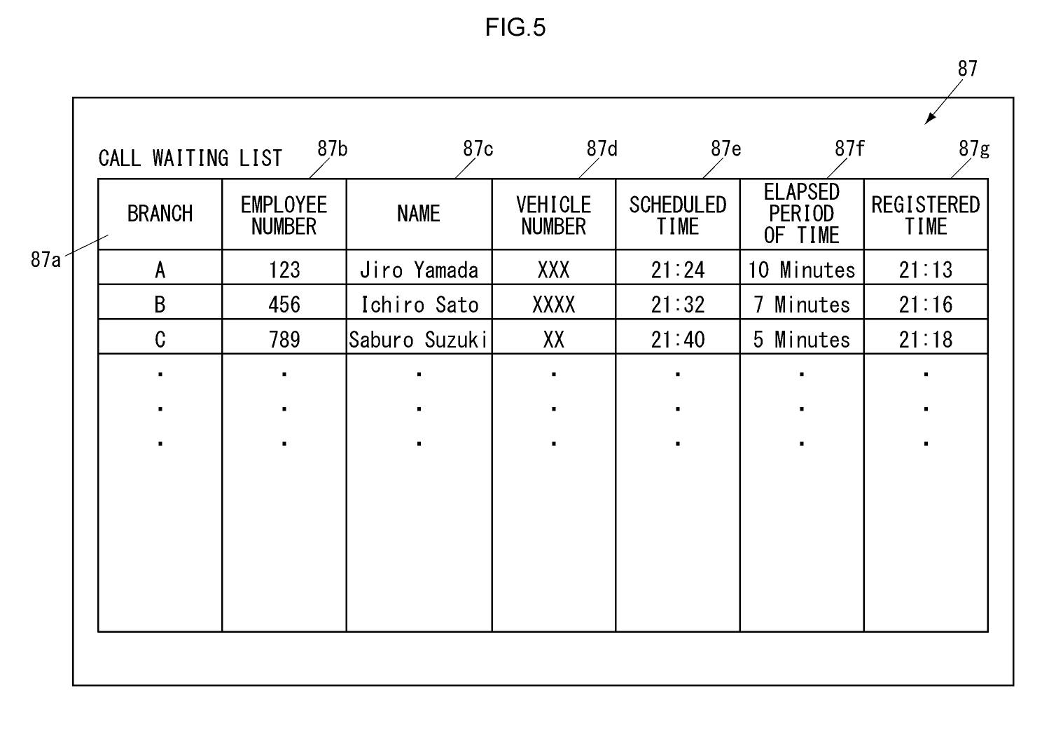

FIG. 5 is a view showing an example of a list screen displayed on the monitor 45 of the to-be-called side device 40.

As shown in FIG. 5, the “present to-be-called person” region 81, the “call manager” region 82, the “to-be-called person” region 83, the “driver's license” region 84, the “registered face image” region 85, and the “past history” region 86 are not shown in the list screen different from the call screen. The list screen is a screen displayed on the monitor 45 of the to-be-called side device 40 which waits for a call.

Next, an operation of the roll call system 10 will be explained.

Note that each time the PC 21 of the call side device 20 acting as the server updates the call waiting list, the PC 21 delivers the call waiting list to the call side devices 20 acting as the clients and to all the to-be-called side devices 40. Accordingly, the same call waiting list is displayed on the monitors 25 of all the call side devices 20 and on the monitors 45 of all the to-be-called side devices 40 at all times.

<Call Request in the to-be-Called Side Device 40>

An operation of the to-be-called side device 40 when a call is requested will be explained.

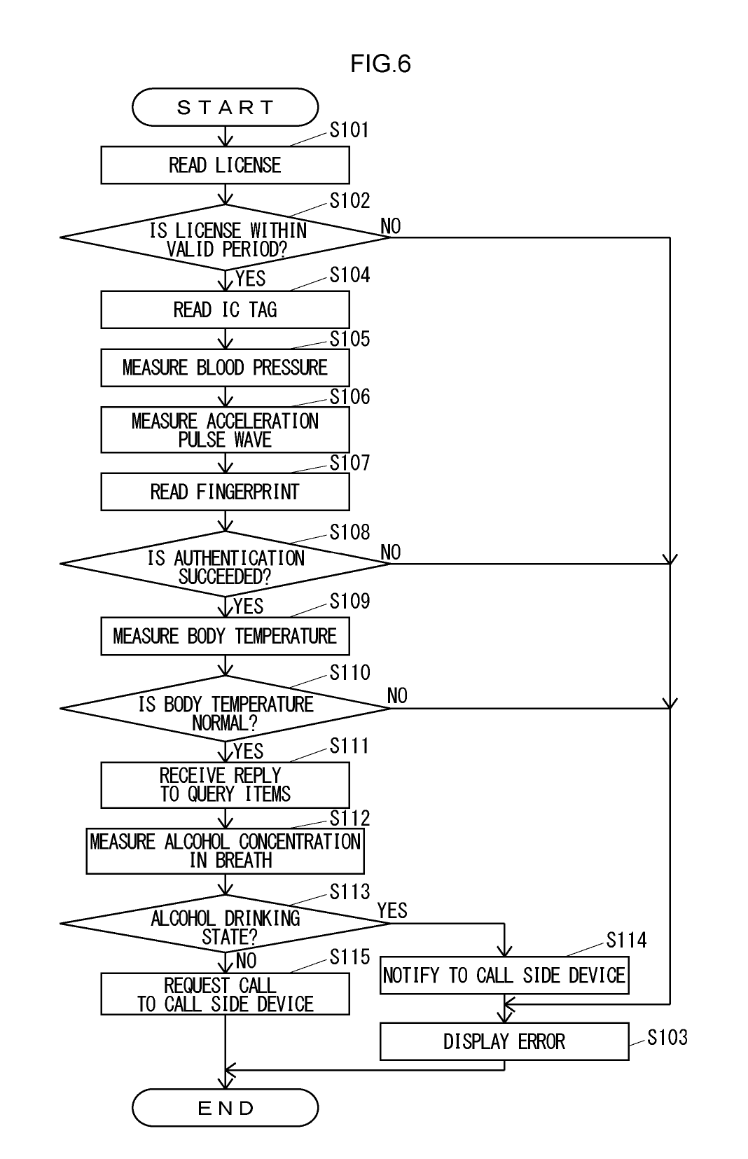

FIG. 6 is a flowchart of the operation of the to-be-called side device 40 when the call is requested.

When a to-be-called person inserts a license into the license reader 50 of the to-be-called side device 40, the PC 41 of the to-be-called side device 40 performs the operation shown in FIG. 6. Note that, while the operation shown in FIG. 6 is performed, the PC 41 sequentially guides and displays the actions to be performed by the to-be-called person on the monitor 45. Accordingly, it is sufficient for the to-be-called person to perform the actions according to the display of the monitor 45.

As shown in FIG. 6, the PC 41 reads information from the license by the license reader 50 (S101) and determines whether or not the license is within a valid period based on the valid period of the license in information read at S101 (S102).

When the PC 41 determines that the license is not within the valid period at S102, the PC 41 makes an error display showing that a call fails on the monitor 45 (S103) and finishes a process shown in FIG. 6. In contrast, when the PC 41 determines that the license is within the valid period at S102, the PC 41 reads information from the IC tag carried by the to-be-called person by the IC tag reader 51 (S104).

Next, the PC 41 measures the blood pressure of the to-be-called person by the blood-pressure gauge 53 (S105) and measures the acceleration pulse wave of the to-be-called person by the acceleration sphygmograph 54 (S106). Note that the value measured by the blood-pressure gauge 53 and the value measured by the acceleration sphygmograph 54 are displayed on the monitor 45 by the PC 41.

Next, the PC 41 reads the fingerprint of the to-be-called person by the fingerprint authentication device 52 (S107) and determines whether or not an authentication by the fingerprint authentication device 52 is succeeded based on the fingerprint read at S107 (S108).

When the PC 41 determines that the authentication by the fingerprint authentication device 52 fails at S108, the PC 41 makes an error display on the monitor 45 showing that the call fails (S103) and finishes the process shown in FIG. 6. In contrast, when the PC 41 determines that the authentication by the fingerprint authentication device 52 is succeeded at S108, the PC 41 measures the body temperature of the to-be-called person by thermometer 55 (S109) and determines whether or not the body temperature measured at S109 is normal (S110). A range of body temperature which is determined normal is previously set, and when the body temperature measured at S109 is within the previously set range, the PC 41 determines that the body temperature measured at S109 is normal. Note that the value measured by thermometer 55 is displayed on the monitor 45 by the PC 41.

When the PC 41 determines that the measured body temperature is abnormal at S110, the PC 41 makes an error display showing that the call fails on the monitor 45 (S103) and finishes the process shown in FIG. 6. In contrast, when the PC 41 determines that the measured body temperature is normal at S110, the PC 41 receives a reply to query items made by the to-be-called person (S111).

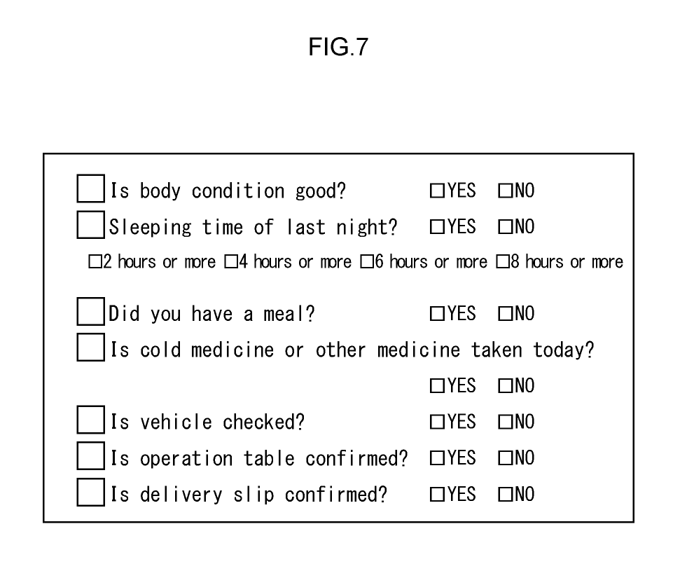

FIG. 7 is a view showing an example of the query items displayed on the monitor 45.

The query items at S111 are query items useful to a call such as query items as to a health state and the like as shown in, for example, FIG. 7. The query items may include various query items such as a degree of stress felt by the to-be-called person, whether or not a drug, an antihypnotic agent, and the like are used in addition to the query items shown in FIG. 7.

The query items are displayed on the monitor 45 and replied by the to-be-called person via the operation unit 46 such as a mouse, a touch panel disposed on the monitor 45, and the like.

As shown in FIG. 6, the alcohol drinking state check means 41a as one of the functions of the PC 41 measures the alcohol concentration in a breath of the to-be-called person by the alcohol measurement unit 56 after S111 (S112). Next, the PC 41 determines whether or not the to-be-called person is in an alcohol drinking state based on the alcohol concentration measured at S112 (S113). The PC 41 is previously set with a threshold value for determining that a to-be-called person is in the alcohol drinking state, and when the alcohol concentration measured at S112 is equal to or more than the threshold value, the PC 41 determines that the to-be-called person is in the alcohol drinking state. Note that the value measured by the alcohol measurement unit 56 is displayed on the monitor 45 by the PC 41.

When the PC 41 determines that the to-be-called person is in the alcohol drinking state at S113, the PC 41 notifies ID information of the to-be-called person such as the name read from the license and the IC tag at S101 and S104 and that the to-be-called person was in the alcohol drinking state to the call side device 20 acting as the server via the communication unit 47 (S114), makes an error display showing that the call fails on the monitor 45 (S103), and finishes the processes shown in FIG. 6.

Note that when the PC 21 of the call side device 20 acting as the server receives that the to-be-called person was in the alcohol drinking state from the to-be-called side device 40, the PC 21 transmits that the to-be-called person was in the alcohol drinking state to a not shown predetermined device as an urgent notification together with ID information of the to-be-called person to whom it is determined by the PC 41 that he or she was in the alcohol drinking state. The predetermined device is the mobile communication terminal, the PC, and the like of a management executive and a manager of a carrier and receives the urgent notification from the call side device 20 acting as the server by phone, E mail, and the like. Note that the urgent notification may be directly received from the to-be-called side device 40 without via the call side device 20 acting as the server.

When the PC 41 determines that the to-be-called person is not in the alcohol drinking state at S113, the PC 41 transmits a call request to the call side device 20 acting as the server via the communication unit 47 (S115) and finishes the process shown in FIG. 6. When the PC 41 transmits the call request, the PC 41 notifies various types of information such as the image of the license read at S101, the identification information of the to-be-called side device 40 itself, the name read from the license at S101, the vehicle number of a truck read from the IC tag at S104, the blood pressure measured at S105, the acceleration pulse wave measured at S106, the body temperature measured at S109, the reply received at S111, the alcohol concentration measured at S112, and the like to the call side device 20 acting as the server together with the call request.

### <Registration to the Call Waiting List in the Call Side Device 20 Acting as the Server>

An operation of the call side device 20 acting as the server when a to-be-called person is registered to the call waiting list will be explained.

The call side device 20 acting as the server shows the call screen shown in FIG. 4 on the monitor 25.

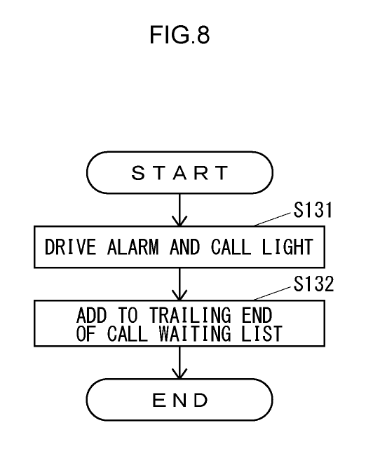

FIG. 8 is a flowchart of the operation of the call side device 20 acting as the server when the to-be-called person is registered to the call waiting list.

When the PC 21 of the call side device 20 acting as the server receives a call request from a to-be-called side device 40, the PC 21 starts a process shown in FIG. 8.

As shown in FIG. 8, the PC 21 drives the alarm 28 and the call light 29 (S131). That is, the PC 21 notifies the reception of the call request to a call manager by the sound output by the alarm 28 and the light emitted by the call light 29.

Next, the PC 21 adds the to-be-called person by whom the received call request is performed to the trailing end of the call waiting list (S132) and finishes the process shown in FIG. 8.

As a result of process at S132, the same call waiting list in which the to-be-called person is updated is displayed on the monitors 25 of all the call side devices 20 and on the monitors 45 of all the to-be-called side devices 40.

<Obtaining of Evidence in the to-be-Called Side Device 40>

An operation of the to-be-called side device 40 when evidence is obtained will be explained.

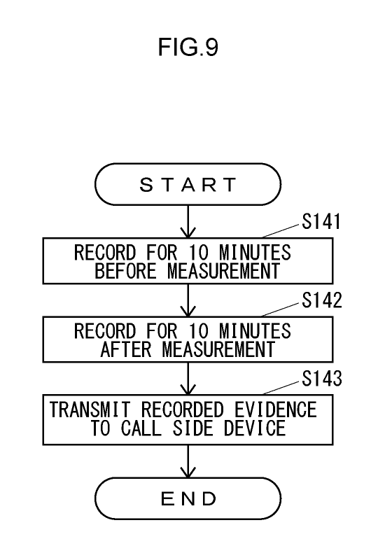

FIG. 9 is a flowchart of the operation of the to-be-called side device 40 when the evidence is obtained.

When the alcohol concentration is measured at S112 shown in FIG. 6, the PC 41 of the to-be-called side device 40 starts a process shown in FIG. 9.

As shown in FIG. 9, the pre-check/post-check record means 41d which is one of the functions of the PC 41 records the data for 10 minutes before the alcohol concentration is measured at S112 in the data which is already recorded to the ring buffer by the temporarily record continuation means 41c to a non-volatile memory as evidence (S141), and subsequently records the data for 10 minutes after the alcohol concentration was measured at S112 in the data which is to be recorded to the ring buffer from that time by the temporarily record continuation means 41c to the non-volatile memory as evidence (S142). When the pre-check/post-check record means 41d records the evidence, the pre-check/post-check record means 41d records the result of measurement of the alcohol concentration and the evidence in relation with each other. The evidence recorded as described above can be used later by being read and reproduced from the PC 41. Note that when the pre-check/post-check record means 41d records the data for 10 minutes after the alcohol concentration was measured to the non-volatile memory as the evidence, the pre-check/post-check record means 41d may directly record the peripheral states obtained by the peripheral state obtaining means 41b in place of the data recorded to the ring buffer by the temporarily record continuation means 41c.

Next, the external transmission means 41e which is one of the functions of the PC 41 transmits the evidence recorded to the non-volatile memory at S141 and S142 to the call side device 20 acting as the server (S143) and finishes the process shown in FIG. 9. When the external transmission means 41e transmits the evidence to the call side device 20 acting as the server, the external transmission means 41e transmits the result of measurement of the alcohol concentration and the evidence in relation with each other. Note that the PC 21 of the call side device 20 acting as the server records the information such as the evidence and the like transmitted from the to-be-called side device 40. The evidence recorded as described above can be used later by being read and reproduced from the PC 21.

### <Performance of a Call in the Call Side Device 20>

An operation of the call side device 20 when a call is performed will be explained.

When no call is started, nothing is displayed in the “present to-be-called person” region 81, the “to-be-called person” region 83, the “driver's license” region 84, the “registered face image” region 85, and the “past history” region 86 in the call screen on the monitor 25 of the call side device 20 (refer to FIG. 4).

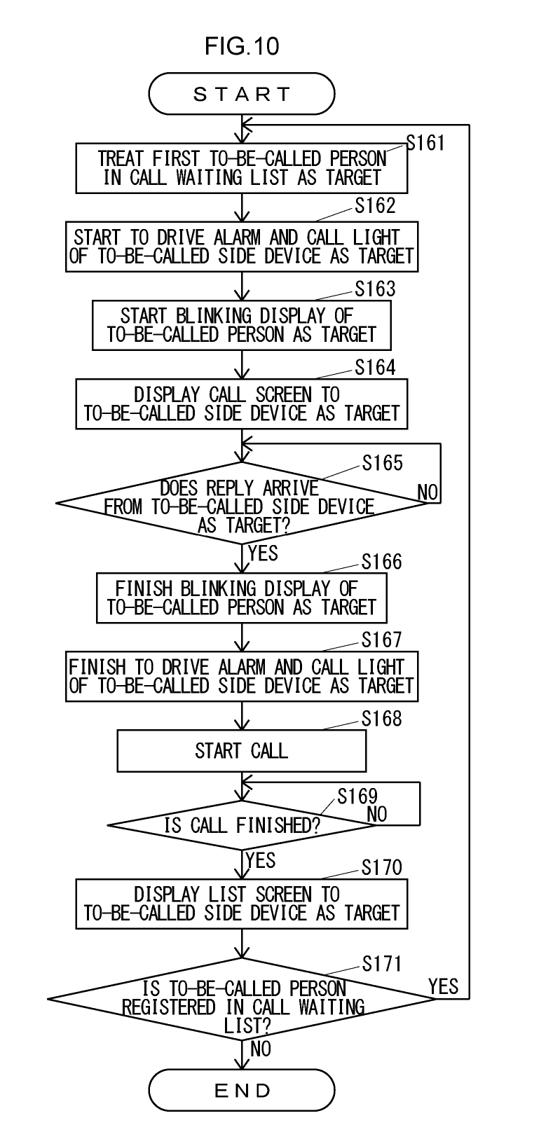

FIG. 10 is a flowchart of an operation of the call side device 20 when a call is performed.

When a call manager instructs to start a call to the call side device 20 by, for example, depressing a call start button from the operation unit 26 of the call side device 20, the PC 21 of the call side device 20 starts a process shown in FIG. 10.

As shown in FIG. 10, the PC 21 treats a first to-be-called person in the call waiting list as a target (S161). At the time, the PC 21 deletes the first to-be-called person in the call waiting list from the call waiting list as well as moves the to-be-called person from the “call waiting list” region 87 to the “present to-be-called person” region 81 in the call screen on the monitor 25. That is, each of the rows of the call waiting list shown in the “call waiting list” region 87 is shifted up one row by moving the first to-be-called person to the “present to-be-called person” region 81. Further, the image, which is transmitted from the to-be-called side device 40 to the call side device 20 acting as the server together with the call request of the to-be-called person as the target, is shown in the “driver's license” region 84 on the monitor 25. Further, the face image and the past history, which are previously registered in the PC 21 of the call side device 20 acting as the server as to the to-be-called person as the target, are shown in the “registered face image” region 85 and the “past history” region 86 respectively.

As a result of process at S161, the same call waiting list in which the to-be-called person is updated is displayed on the monitors 25 of all the call side devices 20 and the monitors 45 of all the to-be-called side devices 40.

Next, the PC 21 starts to drive the alarm 48 and the call light 49 of a to-be-called side device 40 as a target by transmitting an instruction to a to-be-called side device 40 as a target via the network 11 (S162). The to-be-called side device 40 as the target is the to-be-called side device 40 of the to-be-called person who is treated as the target at S161 in all the to-be-called side devices 40 capable of communicating with the call side devices 20. The PC 41 of the to-be-called side device 40 as the target starts to cause the alarm 48 to output sound and the call light 49 to emit light in response to the instruction from the call side device 20. Further, the PC 21 may start to blink the image displayed on the monitor 45 of the to-be-called side device 40 as the target.

Next, the PC 21 starts to blinkingly display the information of a to-be-called person shown in the “present to-be-called person” region 81 in the call screen on the monitor 25 (S163).

Next, the PC 21 causes the monitor 45 of the to-be-called side device 40 as the target to display the call screen by transmitting an instruction to the to-be-called side device 40 as the target via the network 11 (S164). That is, the monitor 25 of the call side device 20 and the monitor 45 of the to-be-called side device 40 as the target display the same image in synchronization with each other.

Next, the PC 21 determines whether or not a reply arrives from the to-be-called side device 40 as the target until the PC 21 receives the replay from the to-be-called side device 40 as the target (S165). Since the to-be-called person as the target can be aware that he or she becomes a target of a call by the sound output by the alarm 48 of the to-be-called side device 40, the light emitted by the call light 49, and the display on the monitor 45, the reply can be transmitted from the operation unit 46 of the to-be-called side device 40 as the target to the call side device 20 as soon as the to-be-called person finishes a preparation for receiving the call.

When the PC 21 determines that the replay arrives from the to-be-called side device 40 as the target at S165, the PC 21 finishes the blink display of the information of the to-be-called person displayed in the “present to-be-called person” region 81 in the call screen on the monitor 25 (S166).

Next, the PC 21 finishes to drive the alarm 48 and the call light 49 of the to-be-called side device 40 as the target by transmitting an instruction to the to-be-called side device 40 as the target via the network 11 (S167). The PC 41 of the to-be-called side device 40 as the target finishes the sound output by the alarm 48 and the light emitted by the call light 49 in response to the instruction from the call side device 20. Note that when the PC 21 starts to blink the image displayed on the monitor 45 of the to-be-called side device 40 as the target at S162, the PC 21 also finishes the blink of the image.

Next, the PC 21 permits the call manager to perform the call to the to-be-called person by communicating with the to-be-called side device 40 as the target via the network 11 (S168). Specifically, the sound input from the microphone 42 of the to-be-called side device 40 as the target is output from the speaker 24 of the call side device 20 via the network 11. The sound input from the microphone 22 of the call side device 20 is output from the speaker 44 of the to-be-called side device 40 as the target via the network 11. The image picked up by the video camera 43 of the to-be-called side devices 40 as the target is shown in the “to-be-called person” region 83 on the monitor 45 of the to-be-called side device 40 as the target as well as shown also in the “to-be-called person” region 83 on the monitor 25 of the call side device 20 via the network 11. Further, the image picked up by the video camera 23 of the call side device 20 is shown in the “call manager” region 82 on the monitor 25 of the call side device 20 as well as shown also in the “call manager” region 82 on the monitor 45 of the to-be-called side device 40 as the target via the network 11. As described above, since the call manager can perform the call while confirming the appearance of the to-be-called person and making dialog with the to-be-called person, the call manager can perform the call while feeling the physical condition and the like of the to-be-called person from the way of dialog, the voice, the facial color, the eyes, and the like of the to-be-called person.

Next, the PC 21 determines whether or not the call is finished until the PC 21 determines that the call is finished (S169). The call manager can instruct to finish the call to the call side device 20 by, for example, depressing a call finish button from the operation unit 26 of the call side device 20, and the like.

When the PC 21 determines that the call is finished at S169, the PC 21 causes the monitor 45 of the to-be-called side device 40 as the target to display the list screen by transmitting an instruction to the to-be-called side device 40 as the target via the network 11 (S170).

Next, the PC 21 determines whether or not a to-be-called person is still registered in the call waiting list (S171).

When the PC 21 determines that a to-be-called person is still registered in the call waiting list at S171, the PC 21 returns to the process at S161 again. That is, the PC 21 permits the call to be performed via the to-be-called side devices 40 in the order of the to-be-called persons in the call waiting list.

When the PC 21 determines that no to-be-called person is registered in the call waiting list at S171, the PC 21 finishes the process shown in FIG. 10.

Note that, in the above description, although the example, in which the computers of the call side devices 20 and the to-be-called side devices 40 are composed of personal computers, is shown, the computers of at least ones of the call side devices 20 and the to-be-called side devices 40 may be composed of computers other than the personal computers. For example, the computers of the call side devices 20 and the to-be-called side devices 40 may be composed of mobile communication devices such as mobile phones and the like.

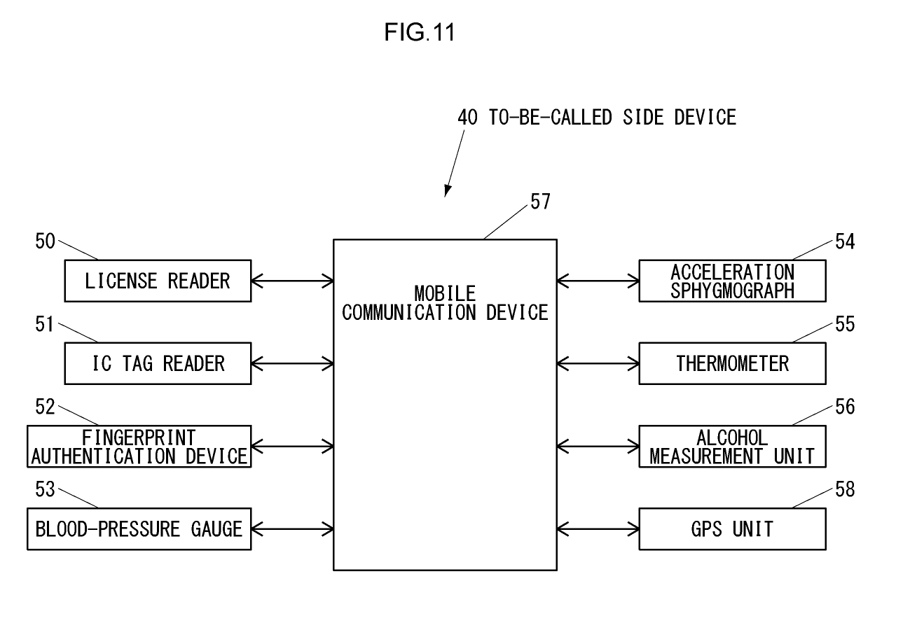

FIG. 11 is a block diagram of the to-be-called side device 40 when the computer is composed of a mobile communication device.

As shown in FIG. 11, the to-be-called side device 40 includes a mobile communication device 57 which is a computer for controlling the to-be-called side devices 40 in its entirety and a GPS (Global Positioning System) unit 58 for obtaining a present position. Further, the to-be-called side device 40 shown in FIG. 11 includes a license reader 50, an IC tag reader 51, a fingerprint authentication device 52, a blood-pressure gauge 53, an acceleration sphygmograph 54, a thermometer 55, and an alcohol measurement unit 56 likewise the example shown in FIG. 3.

Note that, the to-be-called side device 40 shown in FIG. 11 substitutes a microphone, a video camera, a speaker, a monitor, an operation unit, and a communication unit of the mobile communication device 57 for the configuration corresponding to the microphone 42, the video camera 43, the speaker 44, the monitor 45, the operation unit 46, and the communication unit 47 shown in FIG. 3. Further, the to-be-called side device 40 shown in FIG. 11 substitutes the speaker and the monitor of the mobile communication device 57 for the configuration corresponding to the alarm 48 and the call light 49 shown in FIG. 3.

When the to-be-called side device 40 is composed of the mobile communication device 57 as shown in FIG. 11, the to-be-called side device 40 can be easily mounted on a truck. The truck on which the to-be-called side device 40 is mounted becomes a mobile branch of a carrier. When the to-be-called side device 40 is composed of the mobile communication device 57 as shown in FIG. 11 and transmits a call request to the call side device 20, the present position obtained by the GPS unit 58 is also notified to the call side device 20 together with the call request.

As described above, since the to-be-called side device 40 records the peripheral states for 10 minutes before and 10 minutes after the alcohol drinking state check of a to-be-called person who is the user of the to-be-called side device 40 itself in the respective checks, when a communication as to illegality for the alcohol drinking state check is performed for the 10 minutes before and the 10 minutes after the alcohol drinking state check, the communication can be kept as evidence.

Further, since the to-be-called side device 40 transmits the evidence to the call side device 20 acting as the server, the call side device 20 acting as the server can store the evidence. Accordingly, the to-be-called side device 40 can make it difficult to illegally erase the evidence in comparison with the case that the evidence is stored only in the to-be-called side device 40.

Note that, in the roll call system 10 of the embodiment, although all the call side devices 20 are directly used by the call managers, the call side device 20 acting as the server may be in charge of only the management of the call waiting list and only the call side devices 20 acting as the clients may be directly used by the call managers. In the case, the standard waiting time is in inverse proportion to the number of the call side devices 20 acting as the clients.

Although the roll call system 10 of the embodiment includes the plural call side devices 20, the roll call system 10 may include only one call side device 20.

Further, although the roll call system 10 of the embodiment is introduced by the carrier, the user of the roll call system 10 is not limited to the carrier.

Note that, in the roll call system 10 of the embodiment, although the CPU executing the alcohol drinking state check program achieves the alcohol drinking state check means, the peripheral state obtaining means, the temporary record continuation means, the pre-check/post-check record means, and the external transmission means, the respective means may be achieved by mutually different hardware. The respective means are not limited to any specific combination of hardware circuitry and software.

What is claimed is:

1. An alcohol drinking state check device comprising:
alcohol drinking state check means for performing an alcohol drinking state check of a user;
peripheral state obtaining means for obtaining a peripheral state by at least one of sound and image to keep a communication as to the check which is illegal between a person who monitors the check performed by the user and the user as evidence;
temporary record continuation means for continuing the temporary record of the peripheral states obtained by the peripheral state obtaining means; and
pre-check/post-check record means for recording the peripheral states for a predetermined time before and after the check in the respective checks,
wherein the pre-check/post-check record means obtains the record of the peripheral state at least before the check from the record by the temporarily record continuation means in the record of the peripheral states for the predetermined time before and after the check.

2. The alcohol drinking state check device according to claim 1 further comprising external transmission means for transmitting the peripheral states recorded by the pre-check/post-check record means to the outside.

3. A non-transitory computer-readable storage medium storing an alcohol drinking state check program for causing a computer to execute a process comprising:
an alcohol drinking state check step of performing an alcohol drinking state check of a user;
a peripheral state obtaining step of obtaining a peripheral state by at least one of sound and image to keep a communication as to the check which is illegal between a person who monitors the check performed by the user and the user as evidence;
a temporary record continuation step of continuing the temporary record of the peripheral states obtained by the peripheral state obtaining step; and
a pre-check/post-check record step of recording the peripheral states for a predetermined time before and after the check in the respective checks,
wherein the pre-check/post-check record step obtains the record of the peripheral state at least before the check from the record by the temporarily record continuation step in the record of the peripheral states for the predetermined time before and after the check.

4. The non-transitory computer-readable storage medium according to claim 3, wherein the process further comprises an external transmission step of transmitting the peripheral states recorded by the pre-check/post-check record step to the outside.

5. An alcohol drinking state check method comprising:
an alcohol drinking state check step of performing an alcohol drinking state check of a user;
a peripheral state obtaining step of obtaining a peripheral state by at least one of sound and image to keep a communication as to the check which is illegal between a person who monitors the check performed by the user and the user as evidence;
a temporary record continuation step of continuing the temporary record of the peripheral states obtained by the peripheral state obtaining step; and
a pre-check/post-check record step of recording the peripheral states for a predetermined time before and after the check in the respective checks,
wherein the pre-check/post-check record step obtains the record of the peripheral state at least before the check from the record by the temporarily record continuation step in the record of the peripheral states for the predetermined time before and after the check.

6. The alcohol drinking state check method according to claim 5 further comprising an external transmission step of transmitting the peripheral states recorded by the pre-check/post-check record step to the outside.
